(19) United States
(12) Patent Application Publication
(10) Pub. No.: US 2018/0128809 A1
(43) Pub. Date: May 10, 2018

(54) Gas Detection Apparatus and Detection Assembly Thereof

(71) Applicant: METAL INDUSTRIES RESEARCH & DEVELOPMENT CENTRE, Kaohsiung City (TW)

(72) Inventors: Kuan-Chiao Cheng, Kaohsiung City (TW); Chun-Chieh Tseng, Kaohsiung City (TW); Ying-Cheng Lu, Kaohsiung City (TW)

(21) Appl. No.: 15/343,334

(22) Filed: Nov. 4, 2016

(51) Int. Cl. G01N 33/497; A61B 5/097

(52) U.S. Cl. CPC G01N 33/497; G01N 2033/4975; A61B 5/097

(57) ABSTRACT

A gas detection apparatus includes a housing, a detection assembly, and a data acquiring module. The housing includes a chamber, an air inlet, and an air outlet. The air inlet and the air outlet are intercommunicated with the chamber. The detection assembly is mounted in the chamber of the housing and includes a substrate, a detection unit, and a signal transmission port. The detection unit includes at least one specific gas detection port. A surface of the at least one specific gas detection port is covered with a polymer specific gas detection film that has an affinity for a specific gas. The signal transmission port is disposed on the substrate and is electrically connected to the detection unit. The data acquiring module is electrically connected to the signal transmission port of the detection assembly. Thus, a better balance between the production cost and the detection accuracy is achieved.

### BACKGROUND

### 1. Technical Field

The present disclosure relates to a gas detection apparatus and a detection assembly thereof and, more particularly, to a gas detection apparatus capable of detecting the type and content of a specific gas and a detection assembly thereof.

### 2. Description of the Related Art

According to medical research, the gas exhaled by a person carries messages indicating the performance of physical function. When a body suffers from disorders, the type and content of the volatile organic compounds (VOCs) contained in the exhaled gas are changed. Thus, detection of the type and content of specific volatile organic compounds helps health care workers determine the type and degree of disorders.

Determining the type of disorders through detection of the composition of the exhaled gas has been done for years. Many hospitals and medical centers are equipped with gas detection apparatuses with the above function. A person going to such a hospital can find out disease sources in his or her body by using the gas detection apparatuses.

However, the gas detection apparatuses are generally made by complicated processes and expensive components to achieve higher detection accuracy and, thus, are difficult to produce and costly. For ordinary people, the gas detection apparatuses cannot be easily acquired and are not an easy burden. In an example disclosed in U.S. Pat. No. 6,712,770, a breath-based diagnostic device includes a multi-sensor array having piezoelectric quartz crystals coated with a synthetic peptide for the diagnosis of uremia. However, the synthetic peptide is complicated to produce and is not easy to acquire. China Patent Publication No. CN104677958 discloses an alcohol sensor including a plurality of detection layers. The alcohol sensor must be produced by magnetron sputtering, thin film deposition, etc. and have the same disadvantages of difficulties in production and acquiring. If people cannot use the gas detection apparatus to proceed with self-examination at home, an undetected minor disorder could become an incurable disease. The rushing toil resulting from seeking medical treatment further accelerate weakening of the spirit. These problems are caused by the failure in a better balance between the production cost and the detection accuracy of the conventional gas detection apparatuses.

Thus, a need exist for a novel gas detection apparatus and a detection assembly thereof to solve the problems caused by the failure in a better balance between the production cost and the detection accuracy of the conventional gas detection apparatuses.

### SUMMARY

An objective of the present disclosure is to provide a gas detection apparatus and a detection assembly thereof. The gas detection apparatus can detect the type and content of a specific gas through the detection assembly and can have a lower production cost while maintaining the detection accuracy, obtaining a better balance between the production cost and the detection accuracy.

As used herein, the term “electrical connection” refers to wire or wireless connection or a combination thereof (such as heterogeneous network) between two devices to enable transmission of signals or electricity between the two devices.

To achieve the above objective, a gas detection apparatus is provided for detecting a specific gas contained in a gas exhaled from a human body and includes a housing, a detection assembly, and a data acquiring module. The housing includes a chamber, an air inlet, and an air outlet. The air inlet and the air outlet are intercommunicated with the chamber. The detection assembly is mounted in the chamber of the housing and includes a substrate, a detection unit, and a signal transmission port. The detection unit includes at least one specific gas detection port. A surface of the at least one specific gas detection port is covered with a polymer specific gas detection film that has an affinity for a specific gas. The signal transmission port is disposed on the substrate and is electrically connected to the detection unit. The data acquiring module is electrically connected to the signal transmission port of the detection assembly. The gas detection apparatus can detect the type and content of the specific gas through simple disposition of the polymer specific gas detection film, thereby reducing the production cost while maintaining the detection accuracy. Thus, a better balance between the production cost and the detection accuracy is achieved.

In another aspect, a detection assembly is provided and includes a substrate, a detection unit, and a signal transmission port. The detection unit includes at least one specific gas detection port for a specific gas. A surface of the at least one specific gas detection port is covered with a polymer specific gas detection film that has an affinity for the specific gas. The signal transmission port is disposed on the substrate and is electrically connected to the detection unit. The detection assembly can detect the type and content of the specific gas through simple disposition of the polymer specific gas detection film, thereby reducing the production cost while maintaining the detection accuracy. Thus, a better balance between the production cost and the detection accuracy is achieved.

In an example, the polymer specific gas detection film is a composite of a polymer compound and an electrically conductive material. Thus, the electrical conductivity of the polymer specific gas detection film can be increased by the electrically conductive material to increase the electrical connection effect between the polymer specific gas detection film and the specific gas detection port, such that the signal outputted by the signal transmission port can more clearly present the resistance value information of the detection unit. Furthermore, the polymer specific gas detection film can be simply formed by a polymer composite graphene solution to simplify the production procedure of the detection assembly, increasing the detection accuracy and reducing the production cost.

In another example, the polymer specific gas detection film includes a polymer layer and an electrically conductive layer. The polymer layer is formed by a polymer compound. The electrically conductive layer is formed by an electrically conductive material. The electrically conductive layer has a surface coupled to a surface of the polymer layer. Thus, the electrical conductivity of the polymer specific gas detection film can be increased by the electrically conductive material to increase the electrical connection effect between the polymer specific gas detection film and the specific gas detection port, such that the signal outputted by the signal transmission port can more clearly present the resistance value information of the detection unit. Furthermore, the polymer layer can be formed on the outermost portion for reacting with a gas to be detected, and the electrical connection between the electrically conductive layer and the specific gas detection port further permits the signal outputted by the signal transmission port to more clearly present the reaction result of the polymer layer, further increasing the detection accuracy.

In an example, the polymer compound is a compound including a structure of thiophene, aniline, or ethylenediamine, and the electrically conductive material is graphene. Thus, a user can determine the type and content of the specific gas according to an extent of the change in the resistance value of the polymer specific gas detection film to obtain a qualitative (the type of the gas) and quantitative (the content of the gas) detection result, increasing the detection accuracy.

In an example, the detection unit further includes at least one normal respiratory gas detection port having a surface covered by a polymer normal respiratory gas detection film having an affinity for a normal respiratory gas. Thus, the detection assembly can detect the type and content of the normal respiratory gas through simple disposition of the polymer normal respiratory gas detection film, thereby reducing the production cost while maintaining the detection accuracy. Thus, a better balance between the production cost and the detection accuracy is achieved.

In an example, the polymer normal respiratory gas detection film is formed by a compound having a structure of pyrrolidone or thiophene. Thus, a user can determine the type and content of the normal respiratory gas according to an extent of the change in the resistance value of the polymer normal respiratory gas detection film to obtain a qualitative (the type of the gas) and quantitative (the content of the gas) detection result, increasing the detection accuracy.

In an example, the detection unit further includes at least one slow reaction detection port covered by a slow reaction detection film not reacting with the normal respiratory gas and the specific gas. Thus, the detection assembly can determine the presence of the specific gas and the normal respiratory gas by simple disposition of the slow reaction detection film, thereby reducing the production cost while maintaining the detection accuracy. Thus, a better balance between the production cost and the detection accuracy is achieved.

In an example, a reference point is defined in a surface of the substrate, and the detection unit has a center located on the reference point and is disposed on the substrate in a circumferential direction. Thus, a gas to be detected can evenly pass through the annularly disposed detection unit, increasing the detection accuracy.

In an example, the air inlet of the housing is aligned with the reference point in a direction perpendicular to the surface of the substrate. Thus, a gas to be detected can evenly pass through the annularly disposed detection unit, increasing the detection accuracy.

The present disclosure will become clearer in light of the following detailed description of illustrative embodiments of the present disclosure described in connection with the drawings.

### BRIEF DESCRIPTION OF THE DRAWINGS

FIG. 1 is a perspective of a gas detection apparatus according to the present disclosure.

FIG. 2 is a top view of a detection assembly of the gas detection apparatus according to the present disclosure.

FIG. 3 is a diagrammatic cross section view taken along section line A-A of FIG. 2, illustrating a structure pattern of a detection film of the detection assembly according to the present disclosure.

FIG. 4 is a diagrammatic cross sectional view illustrating another structure pattern of the detection film of the detection assembly according to the present disclosure.

### DETAILED DESCRIPTION

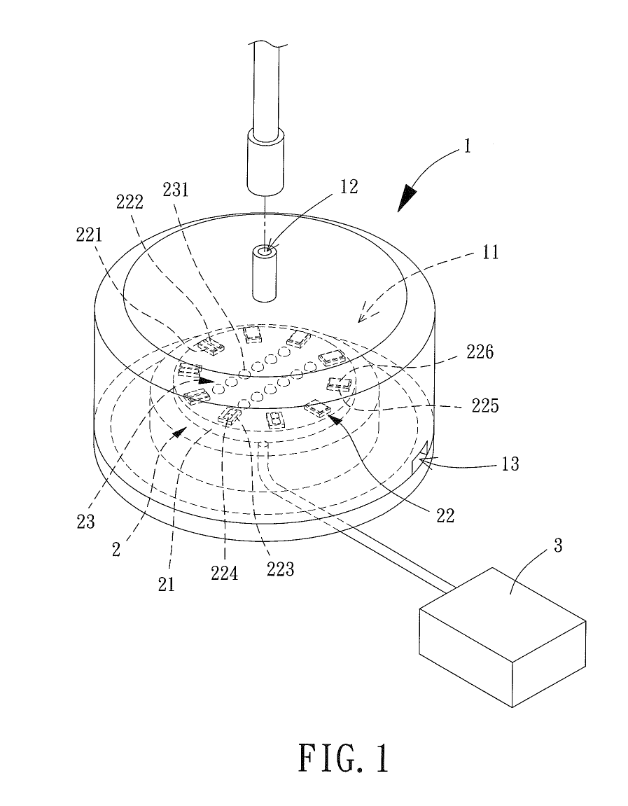

With reference to FIG. 1, a gas detection apparatus according to the present disclosure includes a housing 1, a detection assembly 2, and a data acquiring module 3. The detection assembly 2 is mounted in the housing 1. The data acquiring module 3 is electrically connected to the detection assembly 2.

The housing 1 includes a chamber 11, an air inlet 12, and an air outlet 13. The air inlet 12 and the air outlet 13 are intercommunicated with the chamber 11.

The detection assembly 2 is mounted in the chamber 11 of the housing 1 and includes a substrate 21, a detection unit 22, and a signal transmission port 23. The detection unit 22 includes at least one specific gas detection port 221. The at least one specific gas detection port 221 includes a surface covered with a polymer specific gas detection film 222 having an affinity for a specific gas. The signal transmission port 23 is disposed on the substrate 21 and is electrically connected to the detection unit 22. The substrate 21 can be a printed circuit board, and the polymer material is formed by a compound having a higher molecular mass, which can be appreciated by a person having ordinary skill in the art.

The specific gas detection port 221 is formed by an electrically conductive material and has a fixed resistance. Furthermore, the polymer specific gas detection film 222 is a compound including a structure of thiophene, aniline, or ethylenediamine, and the specific gas is butyric acid, ethymercaptan, and ammonia. Thus, when the specific gas detection port 221 is covered by the polymer specific gas detection film 222, since the polymer specific gas detection film 222 changes its resistance value at the time contacting the specific gas, the specific gas detection port 221 and the polymer specific gas detection film 222 of the detection unit 22 are equivalent to a fixed resistance and a variable resistance. Furthermore, when the detection unit 22 is electrically connected to the signal transmission port 23, the signal outputted by the signal transmission port 23 can include the resistance value information of the detection unit 22, and the resistance value information can be used to obtain an extent of the change of the resistance value of the polymer specific gas detection film 222 by using a partial pressure formula, maintaining the detection accuracy.

The detection unit 22 can further include at least one normal respiratory gas detection port 223 having a surface covered by a polymer normal respiratory gas detection film 224 having an affinity for a normal respiratory gas. In a case that the normal respiratory gas is oxygen, the polymer normal respiratory gas detection film 224 is a polymer compound having a pyrrolidone structure. Alternatively, when the normal respiratory gas is nitrogen, the polymer normal respiratory gas detection film 224 is a polymer compound having a thiophene structure. Thus, when the detection unit 22 has both of the specific gas detection port 221 and the normal respiratory gas detection port 223, the polymer specific gas detection film 222 and the polymer normal respiratory gas detection film 224 have different properties and can respectively react with the specific gas and the normal respiratory gas. A user can simultaneously obtain the extent of the change of the resistance values of the polymer specific gas detection film 222 and the polymer normal respiratory gas detection film 224 and can determine the type and content of the specific gas according to the extent of the change to obtain a qualitative (the type of the gas) and quantitative (the content of the gas) detection result, increasing the detection accuracy.

Furthermore, the detection unit 22 can further include at least one slow reaction detection port 225 covered by a slow reaction detection film 226 not reacting with the normal respiratory gas and the specific gas. Thus, when the detection unit 22 includes all of the specific gas detection port 221, the normal respiratory gas detection port 223, and slow reaction detection port 225, the polymer specific gas detection film 222 and the polymer normal respiratory gas detection film 224 have different properties and can respectively react with the specific gas and the normal respiratory gas. Furthermore, it is assured that the slow reaction detection film 226 will not react with the specific gas and the normal respiratory gas. The user not only can obtain the extent of the change in the resistance values of the polymer specific gas detection film 222 and the polymer normal respiratory gas detection film 224 but can determine the detected gas is the specific gas or the normal respiratory gas according to the reaction of the slow reaction detection film 226. Furthermore, the extent of the change can be used to determine the type and content of the specific gas to obtain a qualitative (the type of the gas) and quantitative (the content of the gas) detection result, increasing the detection accuracy.

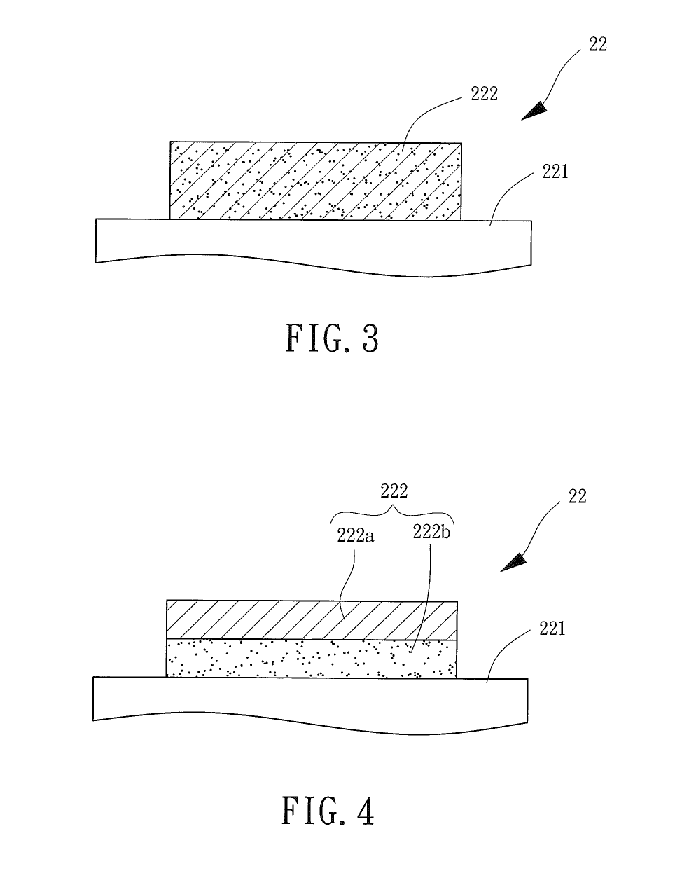

Furthermore, the polymer specific gas detection film 222, the polymer normal respiratory gas detection film 224, and the slow reaction detection film 226 can be formed by the polymer compound or formed by the polymer compound and an electrically conductive material. With reference to FIG. 3, taking the polymer specific gas detection film 222 as an example, the polymer specific gas detection film 222 can be a composite of the polymer compound and the electrically conductive material. As used herein, the term “composite” refers to mixing of two materials in a liquid state. Specifically, the electrically conductive material can be graphene. After the polymer compound is dissolved in an organic solvent, the solution including the polymer compound is mixed with an aqueous solution of graphene to form a polymer composite graphene solution. The polymer composite graphene solution can be dripped attached to a surface of the specific gas detection port 221, and the polymer specific gas detection film 222 can be formed after having been placed steadily for a period of time. Thus, the electrical conductivity of the polymer specific gas detection film 222 can be increased by the electrically conductive material to increase the electrical connection effect between the polymer specific gas detection film 222 and the specific gas detection port 221, such that the signal outputted by the signal transmission port 23 can more clearly present the resistance value information of the detection unit 22. Furthermore, the polymer specific gas detection film 222 can be simply formed by the polymer composite graphene solution to simplify the production procedure of the detection assembly 2, increasing the detection accuracy and reducing the production cost.

With reference to FIG. 4, in an alternative example, the polymer specific gas detection film 222 includes a polymer layer 222a and an electrically conductive layer 222b. The polymer layer 222a is formed by a polymer compound, and the electrically conductive layer 222b is formed by an electrically conductive material. The electrically conductive layer 222b has a side coupled to the specific gas detection port 221. The other side of the electrically conductive layer 222b is coupled to the polymer layer 222a. Specifically, the electrically conductive material can be graphene. A solution including graphene is firstly dripped attached to a surface of the specific gas detection port 221. After the solution including graphene forms the electrically conductive layer 222b, a solution including the polymer compound is dripped attached to a surface of the electrically conductive layer 222b to form the polymer layer 222a after having been placed steadily for a period of time. Thus, the electrical conductivity of the polymer specific gas detection film 222 can be increased by the electrically conductive material to increase the electrical connection effect between the polymer specific gas detection film 222 and the specific gas detection port 221, such that the signal outputted by the signal transmission port 23 can more clearly present the resistance value information of the detection unit 22, increasing the detection accuracy. Furthermore, the polymer layer 222a can be formed on the outermost portion for reacting with a gas to be detected, and the electrical connection between the electrically conductive layer 222b and the specific gas detection port 221 further permits the signal outputted by the signal transmission port 23 to more clearly present the reaction result of the polymer layer 222a, further increasing the detection accuracy.

It is noted that the structural pattern of the polymer compound and the electrically conductive material is not limited to the polymer specific gas detection film 222. The polymer normal respiratory gas detection film 224 and the slow reaction detection film 226 also can have the above structural pattern. Furthermore, the way the polymer normal respiratory gas detection film 224 and the slow reaction detection film 226 form the above structural pattern and the coupling relationship are similar to the polymer specific gas detection film 222, and the same effect is provided. Thus, redundant description is not required.

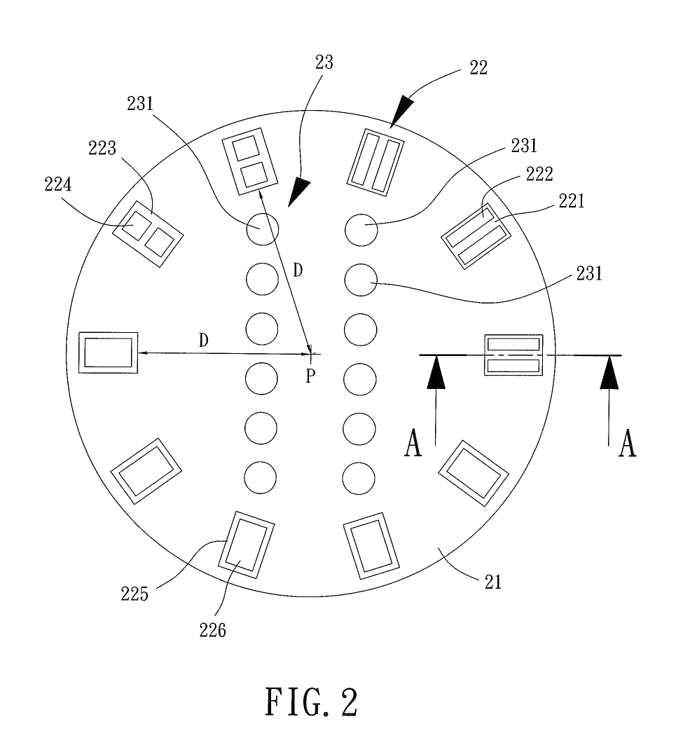

Still referring to FIG. 2, a reference point P is defined in a surface of the substrate 21. The detection unit 22 and the reference point P have a fixed spacing D therebetween. In this embodiment, the detection unit 22 has a center located on the reference point P and is disposed on the substrate 21 in a circumferential direction. Furthermore, the air inlet 12 of the housing 1 is aligned with the reference point P in a direction perpendicular to the surface of the substrate 21. Since the detection unit 22 and the reference point P have a fixed spacing D therebetween and since the air inlet 12 of the housing 1 is aligned with the reference point P, when the gas to be detected enters the chamber 11 of the housing 1 via the air inlet 12, the gas to be detected can evenly flow through the annularly disposed detection unit 22, avoiding adverse influence on the detection result resulting from uneven contact between the polymer specific gas detection film 222 (or the polymer normal respiratory gas detection film 224 and the slow reaction detection film 226) and the gas to be detected. The detection accuracy is, thus, increased.

Furthermore, the air outlet 13 and the air inlet 12 are preferably located on two opposite ends of the housing 1. Furthermore, the air outlet 13 is adjacent to the detection unit 22 of the detection assembly 2. Thus, the gas to be detected flowing into the air inlet 12 can flow toward the detection unit 22 and can flow out via the air outlet 13 adjacent to the detection unit 22. This assures the gas to be detected to reliably contact the polymer specific gas detection film 222 (or the polymer normal respiratory gas detection film 224 and the slow reaction detection film 226) of the detection unit 22, increasing the detection accuracy.

The structural pattern of the signal transmission port 23 is not limited. In this embodiment, the signal transmission port 23 includes a plurality of electrical connections 231 on the substrate 21. Each electrical connection 231 can be electrically connected to a specific gas detection port 221 (or a normal respiratory gas detection port 223 or a slow reaction detection port 225) of the detection unit 22. Alternatively, at least one of the electrical connections 231 is electrically connected to a power source, such that the resistance value information of the detection unit 22 can be transmitted through different electrical connections 231, increasing reliability of data transmission.

The data acquiring module 3 is electrically connected to the signal transmission port 23 of the detection assembly 2. The data acquiring module 3 can receive the resistance value information of the detection unit 22 outputted by the signal transmission port 23. Furthermore, the data acquiring module 3 can analyze the resistance value information through an analytical procedure to obtain the extent of the change in the resistance value of the detection unit 22.

In an example, when the gas detection apparatus is to be used to detect whether a testee has liver cirrhosis, since the contents of butyric acid, ethyl mercaptan, and ammonia in the exhaled gas of a patient having liver cirrhosis are different from those in the exhaled gas of a normal person, the total number of detection ports of the detection unit 22 is 10 (including three specific gas detection ports 221, two normal respiratory gas detection ports 223, and five slow reaction detection ports 225). Nitrogen and oxygen are used as comparative detection items. Furthermore, the polymer compound of the polymer specific gas detection film 222 and the polymer normal respiratory gas detection film 224 can be poly 3-hexylthiophene (P3HT), polyethylenimine (PEI), polyaniline (PANI), poly(3,4-ethylenedioxythiophene) (PEDOT), polyvinylpyrrolidone (PVP), polyethylene (PE), polyethylene glycol (PEG), polyimide (PI), polyvinyl alcohol (PVA), and polytetrafluoroethylene (PTFE). Poly 3-hexylthiophene (P3HT), polyethylenimine (PEI), polyaniline (PANI), poly(3,4-ethylenedioxythiophene) (PEDOT), and polyvinylpyrrolidone (PVP) can respectively react with butyric acid, ethyl mercaptan, ammonia, nitrogen, and oxygen and can change the resistance value of the polymer specific gas detection film 222 or the polymer normal respiratory gas detection film 224. Polyethylene (PE), polyethylene glycol (PEG), polyimide (PI), polyvinyl alcohol (PVA), and polytetrafluoroethylene (PTFE) will not react with butyric acid, ethyl mercaptan, ammonia, nitrogen, and oxygen and will not change the resistance value of the slow reaction detection film 226.

For a detection unit 22 having the above disposition, when a testee exhales toward the air inlet 12, the exhaled gas contacts the detection unit 22, such that the polymer specific gas detection film 222 or the polymer normal respiratory gas detection film 224 has a relative change according to its own polymer material. Through detection of the change in the resistance value of the polymer specific gas detection film 222, the polymer normal respiratory gas detection film 224, and the slow reaction detection film 226, the presence of butyric acid, ethyl mercaptan, ammonia, nitrogen, and oxygen in the type of the gas can be ascertained. Furthermore, through detection of the change in the resistance value of the polymer specific gas detection film 222 and the polymer normal respiratory gas detection film 224, the contents of butyric acid, ethyl mercaptan, ammonia, nitrogen, and oxygen in the gas can be ascertained to obtain a qualitative (the type of the gas) and quantitative (the content of the gas) detection result. The detection result can be used to determine whether the testee has liver cirrhosis. Furthermore, the polymer material mentioned in the above embodiment can only be used to detect whether the testee has liver cirrhosis. When it is desired to use the gas detection apparatus according to the present disclosure to detect other disorders or gases, the polymer compound of the polymer specific gas detection film 222, the polymer normal respiratory gas detection film 224, and the slow reaction detection film 226 can be changed according to different needs. The present disclosure is not restricted in this regard.

In view of the foregoing, the gas detection apparatus and the detection assembly 2 thereof according to the present disclosure can detect the type and content of the specific gas to be detected through simple disposition of the polymer specific gas detection film, thereby reducing the production cost while maintaining the detection accuracy. Thus, a better balance between the production cost and the detection accuracy is achieved.

Thus since the present disclosure disclosed herein may be embodied in other specific forms without departing from the spirit or general characteristics thereof, some of which forms have been indicated, the embodiments described herein are to be considered in all respects illustrative and not restrictive. The scope of the present disclosure is to be indicated by the appended claims, rather than by the foregoing description, and all changes which come within the meaning and range of equivalency of the claims are intended to be embraced therein.

What is claimed is:

1. A gas detection apparatus for detecting a specific gas contained in a gas exhaled from a human body, comprising:
a housing including a chamber, an air inlet, and an air outlet, with the air inlet and the air outlet intercommunicated with the chamber;
a detection assembly mounted in the chamber of the housing and including a substrate, a detection unit, and a signal transmission port, with the detection unit including at least one specific gas detection port, with the at least one specific gas detection port including a surface covered with a polymer specific gas detection film having an affinity for a specific gas, with the signal transmission port disposed on the substrate and electrically connected to the detection unit; and
a data acquiring module electrically connected to the signal transmission port of the detection assembly.

2. The gas detection apparatus as claimed in claim 1, wherein the polymer specific gas detection film is a composite of a polymer compound and an electrically conductive material.

3. The gas detection apparatus as claimed in claim 1, with the polymer specific gas detection film including a polymer layer and an electrically conductive layer, with the polymer layer formed by a polymer compound, with the electrically conductive layer formed by an electrically conductive material, and with the electrically conductive layer having a surface coupled to a surface of the polymer layer.

4. The gas detection apparatus as claimed in claim 2, wherein the polymer compound is a compound including a structure of thiophene, aniline, or ethylenediamine, and the electrically conductive material is graphene.

5. The gas detection apparatus as claimed in claim 1, wherein the detection unit further includes at least one normal respiratory gas detection port having a surface covered by a polymer normal respiratory gas detection film having an affinity for a normal respiratory gas.

6. The gas detection apparatus as claimed in claim 5, wherein the polymer normal respiratory gas detection film is formed by a compound having a structure of pyrrolidone or thiophene.

7. The gas detection apparatus as claimed in claim 5, wherein the detection unit further includes at least one slow reaction detection port covered by a slow reaction detection film not reacting with the normal respiratory gas and the specific gas.

8. The gas detection apparatus as claimed in claim 1, wherein a reference point is defined in a surface of the substrate, and the detection unit has a center located on the reference point and is disposed on the substrate in a circumferential direction.

9. The gas detection apparatus as claimed in claim 8, wherein the air inlet of the housing is aligned with the reference point in a direction perpendicular to the surface of the substrate.

10. A detection assembly comprising:
a substrate;
a detection unit including at least one specific gas detection port, with the at least one specific gas detection port including a surface covered with a polymer specific gas detection film having an affinity for a specific gas; and
a signal transmission port disposed on the substrate and electrically connected to the detection unit.

11. The detection assembly as claimed in claim 10, wherein the polymer specific gas detection film is a composite of a polymer compound and an electrically conductive material.

12. The detection assembly as claimed in claim 10, with the polymer specific gas detection film including a polymer layer and an electrically conductive layer, with the polymer layer formed by a polymer compound, with the electrically conductive layer formed by an electrically conductive material, and with the electrically conductive layer having a surface coupled to a surface of the polymer layer.

13. The detection assembly as claimed in claim 11, wherein the polymer compound is a compound including a structure of thiophene, aniline, or ethylenediamine, and the electrically conductive material is graphene.

14. The detection assembly as claimed in claim 10, wherein the detection unit further includes at least one normal respiratory gas detection port having a surface covered by a polymer normal respiratory gas detection film having an affinity for a normal respiratory gas.

15. The detection assembly as claimed in claim 14, wherein the polymer normal respiratory gas detection film is formed by a compound having a structure of pyrrolidone or thiophene.

16. The detection assembly as claimed in claim 14, wherein the detection unit further includes at least one slow reaction detection port covered by a slow reaction detection film not reacting with the normal respiratory gas and the specific gas.

17. The detection assembly as claimed in claim 10, wherein the air inlet of the housing is aligned with the reference point in a direction perpendicular to the surface of the substrate.
